(19) United States
(12) Patent Application Publication
(10) Pub. No.: US 2025/0115601 A1
(43) Pub. Date: Apr. 10, 2025

(54) PROCESS FOR THE PREPARATION OF FINERENONE AND INTERMEDIATES THEREOF

(71) Applicant: Glenmark Life Sciences Limited, Solapur (IN)

(72) Inventors: Venkata Raghavendra Acharyulu PALLE, Pune (IN); Subbiah RAMAR, Vadodara (IN); Datta SWARUP, Bankura (IN); Shekhar Ashok DESHMUKH, Ahmednagar (IN); Sachin Dasharath VEER, Navi Mumbai (IN); Vijay Gulab GODASE, Sangamner (IN); Pratik PATEL, Vadodara (IN); Gaurav KUMAR, Thane (IN); Mayur JADHAV, Nashik (IN)

(21) Appl. No.: 18/865,825

(22) PCT Filed: May 16, 2023
(86) PCT No.: PCT/IB2023/055003

§ 371 Date: Nov. 14, 2024

(30) Foreign Application Priority Data
May 16, 2022 (IN) 202221027928

(51) Int. Cl. C07D 471/04; C07B 57/00

(52) U.S. Cl. CPC C07D 471/04; C07B 57/00

(57) ABSTRACT

The present invention relates to a process for the preparation of finerenone, a compound of formula I, and intermediates thereof. In particular, the present invention relates to a novel diastereomeric salt of an intermediate of finerenone, a compound of Formula IVa, a process for the preparation thereof, and use thereof in the preparation of finerenone.

### PRIORITY

This application claims the benefit of Indian Provisional Application No. 202221027928 filed on May 16, 2022, entitled “Process for the preparation of finerenone and intermediates thereof”, the contents of which are incorporated herein by reference.

### FIELD OF THE INVENTION

The present invention relates to a process for the preparation of finerenone, a compound of formula I, and intermediates thereof. In particular, the present invention relates to a novel diastereomeric salt of an intermediate of finerenone, a compound of Formula IVa, a process for the preparation thereof, and use thereof in the preparation of finerenone. The present invention also relates to a process for the preparation of (S)-enantiomer of an intermediate compound, the compound of formula IIIa having high chiral purity.

### BACKGROUND OF THE INVENTION

Finerenone, also known by its chemical name (4S)-4-(4-cyano-2-methoxyphenyl)-5-ethoxy-2, 8-dimethyl-1,4-dihydro-1,6-naphthyridine-3-carboxamide is represented by the compound of formula I (hereinafter referred to as the “compound I”).

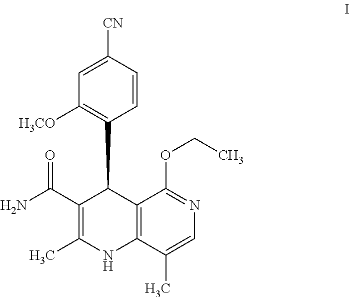 [SMILES: CCOc1ncc(C)c2c1[C@H](c1ccc(C#N)cc1OC)C(C(N)=O)=C(C)N2]

Finerenone is a non-steroidal mineralocorticoid receptor antagonist (MRA), and is marketed in the United States under the trade name of KERENDIA® as tablets in the dosage strength of 10 mg and 20 mg.

Finerenone can be used to reduce the risk of sustained eGFR decline, end stage kidney disease, cardiovascular death, non-fatal myocardial infarction, and hospitalization for heart failure in adult patients with chronic kidney disease (CKD) associated with type-2 diabetes (T2D).

PCT patent publication no. WO 2008/104306 discloses finerenone, the compound I, and a process for its preparation.

Various alternative processes for the preparation of finerenone (the compound I) are reported in the literature. For instance, PCT patent publication no(s). WO2016016287, WO2017032673, WO2019206909, WO2020178175 and WO2021254896 disclose processes for the preparation of finerenone and its intermediates.

### SUMMARY OF THE INVENTION

The present invention relates to a process for the resolution of a racemic mixture of 4-(4-cyano-2-methoxyphenyl)-5-ethoxy-2,8-dimethyl-1,4-dihydro-1,6-naphthyridine-3-carboxylic acid represented by a compound of formula III (the “compound III”),

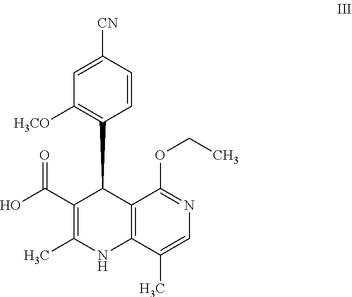 [SMILES: CCOc1ncc(C)c2c1[C@H](c1ccc(C#N)cc1OC)C(C(=O)O)=C(C)N2]

comprising reacting the compound III with a chiral acid to obtain the(S)-enantiomer of 4-(4-cyano-2-methoxyphenyl)-5-ethoxy-2,8-dimethyl-1,4-dihydro-1,6-naphthyridine-3-carboxylic acid represented by the compound of formula IIIa (the “compound IIIa”);

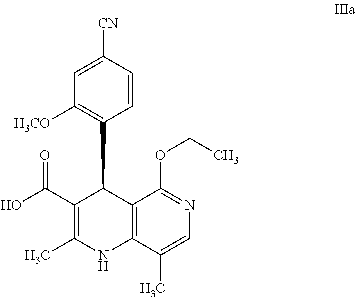 [SMILES: CCOc1ncc(C)c2c1[C@H](c1ccc(C#N)cc1OC)C(C(=O)O)=C(C)N2]

via a diastereomeric salt of the compound III with the chiral acid represented by the compound of formula IV (the “compound IV”),

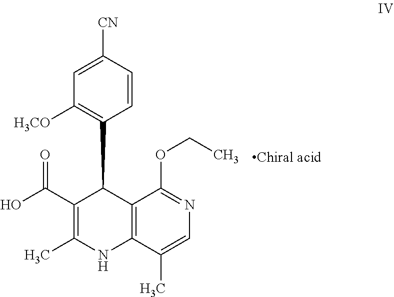 [SMILES: CCOc1ncc(C)c2c1[C@H](c1ccc(C#N)cc1OC)C(C(=O)O)=C(C)N2]

wherein the chiral acid is as defined herein.

The present invention provides a process for the preparation of the(S)-enantiomer of 4-(4-cyano-2-methoxyphenyl)-5-ethoxy-2,8-dimethyl-1,4-dihydro-1,6-naphthyridine-3-carboxylic acid represented by the compound of formula IIIa (the “compound IIIa”);

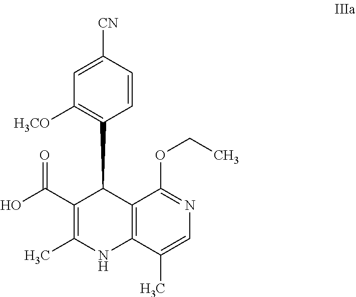 [SMILES: CCOc1ncc(C)c2c1[C@H](c1ccc(C#N)cc1OC)C(C(=O)O)=C(C)N2]

comprising the steps of:
(i) treating a racemic mixture of the compound of formula III (the “compound III”);

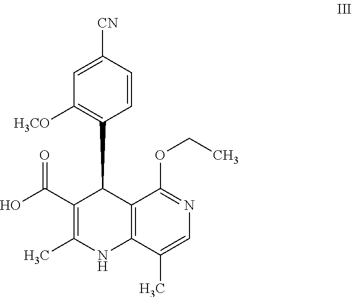 [SMILES: CCOc1ncc(C)c2c1[C@H](c1ccc(C#N)cc1OC)C(C(=O)O)=C(C)N2]

with a chiral acid in a solvent to obtain a diastereomeric salt of compound III with the chiral acid represented by the compound of formula IV (the compound IV);

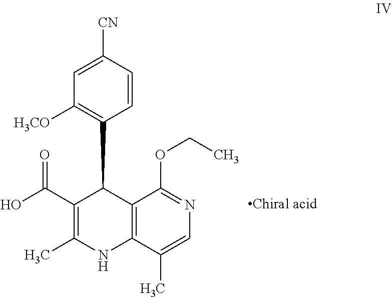 [SMILES: CCOc1ncc(C)c2c1[C@H](c1ccc(C#N)cc1OC)C(C(=O)O)=C(C)N2]

wherein the chiral acid is selected from the group consisting of L-(+) tartaric acid, D-(−) tartaric acid, (+)-dibenzoyl-D-tartaric acid, (−)-dibenzoyl-L-tartaric acid, (+)-di-p-toluoyl-D-tartaric acid, and (−)-di-p-toluoyl-L-tartaric acid; and
(ii) subjecting the compound IV obtained in the step (i) to saponification by treating it with a base or a buffer to obtain the(S)-enantiomer of the compound III, the compound IIIa.

The present invention also relates to a process for the preparation of finerenone (the compound I),

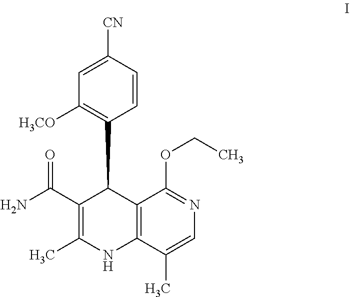 [SMILES: CCOc1ncc(C)c2c1[C@H](c1ccc(C#N)cc1OC)C(C(N)=O)=C(C)N2]

comprising the steps of;
a) obtaining a diastereomeric salt of 4-(4-cyano-2-methoxyphenyl)-5-ethoxy-2,8-dimethyl-1,4-dihydro-1,6-naphthyridine-3-carboxylic acid with a chiral acid represented by the compound of formula IV (the “compound IV”);

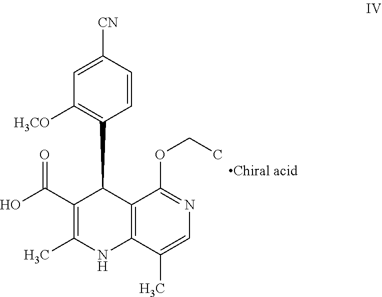 [SMILES: CCOc1ncc(C)c2c1[C@H](c1ccc(C#N)cc1OC)C(C(=O)O)=C(C)N2]

wherein the chiral acid is selected from the group consisting of L-(+) tartaric acid, D-(−) tartaric acid, (+)-dibenzoyl-D-tartaric acid, (−)-dibenzoyl-L-tartaric acid, (+)-di-p-toluoyl-D-tartaric acid, and (−)-di-p-toluoyl-L-tartaric acid;
b) subjecting the compound IV obtained in step (a) to saponification to obtain the(S)-enantiomer of 4-(4-cyano-2-methoxyphenyl)-5-ethoxy-2,8-dimethyl-1,4-dihydro-1,6-naphthyridine-3-carboxylic acid represented by the compound of formula IIIa (the “compound IIIa”);

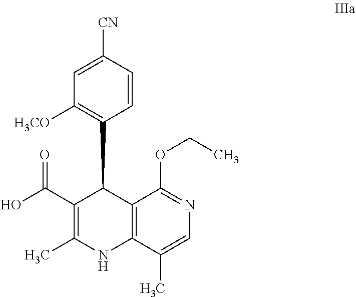 [SMILES: CCOc1ncc(C)c2c1[C@H](c1ccc(C#N)cc1OC)C(C(=O)O)=C(C)N2]

c) converting the compound IIIa obtained in step (b) to finerenone (the compound I); and
d) optionally, purifying the compound I.

The present invention also relates to a diastereomeric salt of 4-(4-cyano-2-methoxyphenyl)-5-ethoxy-2,8-dimethyl-1,4-dihydro-1,6-naphthyridine-3-carboxylic acid with (+)-dibenzoyl-D-tartaric acid represented by the compound of formula IVa (the “compound IVa”);

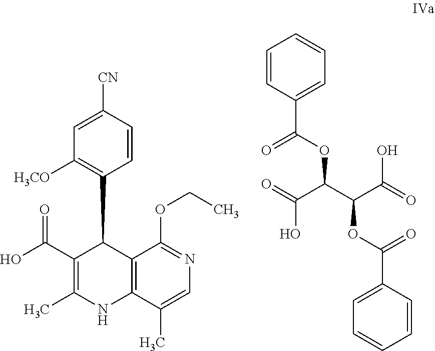 [SMILES: CCOc1ncc(C)c2c1[C@H](c1ccc(C#N)cc1OC)C(C(=O)O)=C(C)N2 ; O=C(O[C@H](C(=O)O)[C@H](OC(=O)c1ccccc1)C(=O)O)c1ccccc1]

The present invention further relates to a process for the preparation of the diastereomeric salt of 4-(4-cyano-2-methoxyphenyl)-5-ethoxy-2,8-dimethyl-1,4-dihydro-1,6-naphthyridine-3-carboxylic acid with (+)-dibenzoyl-D-tartaric acid (the compound IVa); comprising reacting the racemic mixture of the compound III with (+)-dibenzoyl-D-tartaric acid represented by the compound of formula V (the “compound V”),

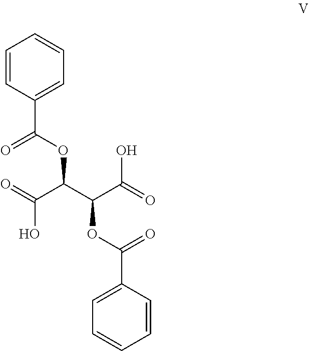 [SMILES: O=C(O[C@H](C(=O)O)[C@H](OC(=O)c1ccccc1)C(=O)O)c1ccccc1]

The present invention further relates to finerenone, the compound I, having chiral purity of greater than 99%, which is obtained by the process of the present invention.

### DETAILED DESCRIPTION OF THE INVENTION

In one aspect, the present invention relates to a process for the resolution of a racemic mixture of 4-(4-cyano-2-methoxyphenyl)-5-ethoxy-2,8-dimethyl-1,4-dihydro-1,6-naphthyridine-3-carboxylic acid represented by the compound of formula III (the “compound III”);

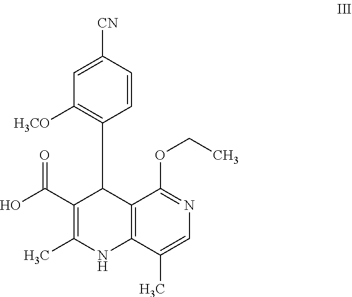 [SMILES: CCOc1ncc(C)c2c1C(c1ccc(C#N)cc1OC)C(C(=O)O)=C(C)N2]

comprising reacting the compound III with a chiral acid to obtain the(S)-enantiomer of 4-(4-cyano-2-methoxyphenyl)-5-ethoxy-2,8-dimethyl-1,4-dihydro-1,6-naphthyridine-3-carboxylic acid represented by the compound of formula IIIa (the “compound IIIa”);

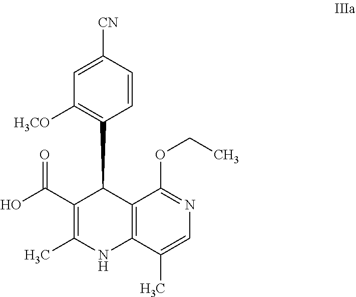 [SMILES: CCOc1ncc(C)c2c1[C@H](c1ccc(C#N)cc1OC)C(C(=O)O)=C(C)N2]

via a diastereomeric salt of the compound III with the chiral acid represented by the compound of formula IV (the “compound IV”);

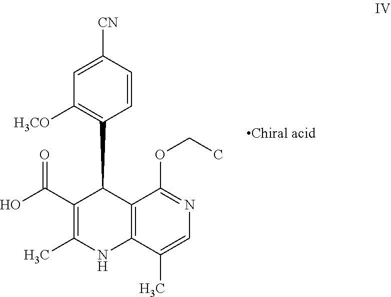 [SMILES: CCOc1ncc(C)c2c1[C@H](c1ccc(C#N)cc1OC)C(C(=O)O)=C(C)N2]

The(S)-enantiomer of 4-(4-cyano-2-methoxyphenyl)-5-ethoxy-2,8-dimethyl-1,4-dihydro-1,6-naphthyridine-3-carboxylic acid, the compound IIIa, is an important intermediate in the preparation of Finerenone (the compound I).

In an embodiment, the resolution of the racemic mixture of the compound III is carried out in a solvent or a mixture of solvents.

The term “racemic mixture” as used herein refers to a mixture of enantiomers which comprises equal amounts of (R)-enantiomer of the compound III and(S)-enantiomer of the compound III, i.e. the molar ratio of the two enantiomers in the racemic mixture is 50:50.

The solvent used in the resolution of the racemic mixture of the compound III is selected from an organic solvent, water or a mixture thereof, wherein the organic solvent includes, but is not limited to, alcohols such as methanol, ethanol, 1,2-ethanediol, methoxyethanol, isopropanol, n-propanol, butanol and the like; hydrocarbons such as toluene, xylene, chlorobenzene, heptane, hexane, cyclohexane and the like; ethers such as dimethyl ether, diethyl ether, diisopropyl ether, tert-butyl methyl ether, dibutyl ether, dimethoxyethane, diethoxyethane, tetrahydrofuran, dioxane and the like; halogenated hydrocarbons such as dichloromethane, dichloroethane, chloroform, ethylene dichloride, and the like; esters such as ethyl acetate, isopropyl acetate, isobutyl acetate, t-butyl acetate and the like; nitriles such as acetonitrile, propionitrile, butyronitrile, benzonitrile and the like; ketone such as acetone, methyl isobutyl ketone, methyl ethyl ketone, cyclohexanone and the like; polar solvent such as dimethylformamide, dimethyl sulfoxide, dimethyl acetamide, or a mixture thereof.

In a further embodiment, the solvent is selected from an organic solvent, water or a mixture thereof; wherein the organic solvent is selected from, but is not limited to, alcohols such as methanol, ethanol, 1,2-ethanediol, methoxyethanol, isopropanol, n-propanol, butanol and the like; ethers such as dimethyl ether, diethyl ether, diisopropyl ether, tert-butyl methyl ether, dibutyl ether, dimethoxyethane, diethoxyethane, tetrahydrofuran (THF), dimethyltetrahydrofuran, dioxane and the like; esters such as ethyl acetate, isopropyl acetate, isobutyl acetate, t-butyl acetate and the like; ketone such as acetone, methyl isobutyl ketone, methyl ethyl ketone, cyclohexanone and the like; polar solvents such as dimethylformamide (DMF), dimethyl sulfoxide, dimethyl acetamide, or a mixture thereof.

In an embodiment, the resolution of the racemic mixture of the compound III with a chiral acid is carried out in a solvent mixture comprising water and an organic solvent selected from methanol, ethanol, 1,2-ethanediol, methoxyethanol, isopropanol, n-propanol, butanol, acetone, tetrahydrofuran (THF), dimethyltetrahydrofuran, dioxane, ethyl acetate, isopropyl acetate, isobutyl acetate, or t-butyl acetate.

In an embodiment, the process for the resolution of the racemic mixture of the compound III is carried out at a temperature ranging from about 20° C. to about 70° C.

In an embodiment, the process for the resolution of the racemic mixture of the compound III to obtain the(S)-enantiomer of the compound III i.e. the compound IIIa involves formation of the diastereomeric salt of compound III with the chiral acid, designated herein as the compound IV.

In an embodiment, the chiral acid used for the resolution of the racemic mixture of the compound III is selected from the group consisting of tartaric acid or its derivatives, (−)-quinic acid, (+)-camphoric Acid, (+)-O-acetyl-L-mandellic Acid, (1R)-(−)-camphor-10-sulphonic acid, (1S)-(−) camphoric acid, (L)-malic acid, S(+)-mandellic acid, R(−)-mandellic acid, and (R)-5-oxopyrrolidine-2-carboxylic acid and the like.

In another embodiment, the chiral acid used for the resolution of the racemic mixture of the compound III is tartaric acid or its derivatives selected from the group consisting of L-(+) tartaric acid, D-(−) tartaric acid, (+)-dibenzoyl-D-tartaric acid, (−)-dibenzoyl-L-tartaric acid, (+)-di-p-toluoyl-D-tartaric acid, and (−)-di-p-toluoyl-L-tartaric acid.

In an embodiment, the chiral acid used for the resolution of the racemic mixture of the compound III is (+)-dibenzoyl-D-tartaric acid.

In an embodiment, the process for the resolution of the racemic mixture of compound III further involves subjecting the diastereomeric salt i.e. the compound IV to saponification in the presence of a base or a buffer to obtain the(S)-enantiomer of 4-(4-cyano-2-methoxyphenyl)-5-ethoxy-2,8-dimethyl-1,4-dihydro-1,6-naphthyridine-3-carboxylic acid, designated herein as the compound IIIa.

In an embodiment, the present invention provides a process for the preparation of (S)-enantiomer of 4-(4-cyano-2-methoxyphenyl)-5-ethoxy-2,8-dimethyl-1,4-dihydro-1,6-naphthyridine-3-carboxylic acid represented by the compound of formula IIIa (the “compound IIIa”);

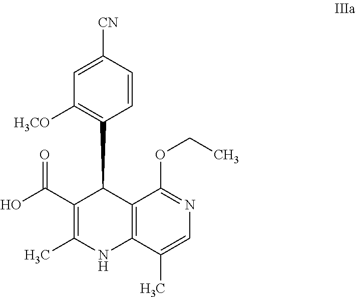 [SMILES: CCOc1ncc(C)c2c1[C@H](c1ccc(C#N)cc1OC)C(C(=O)O)=C(C)N2]

comprising the steps of:
(i) treating a racemic mixture of the compound of formula III (the “compound III”);

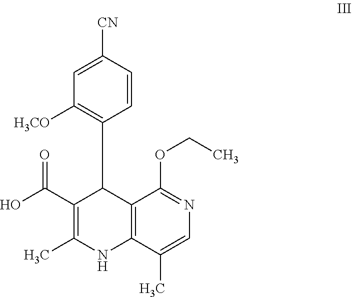 [SMILES: CCOc1ncc(C)c2c1C(c1ccc(C#N)cc1OC)C(C(=O)O)=C(C)N2]

with a chiral acid in a solvent to obtain a diastereomeric salt of compound III with the chiral acid represented by the compound of formula IV (the compound IV);

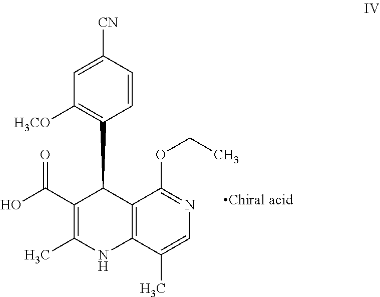 [SMILES: CCOc1ncc(C)c2c1[C@H](c1ccc(C#N)cc1OC)C(C(=O)O)=C(C)N2]

wherein the chiral acid is selected from the group consisting of L-(+) tartaric acid, D-(−) tartaric acid, (+)-dibenzoyl-D-tartaric acid, (−)-dibenzoyl-L-tartaric acid, (+)-di-p-toluoyl-D-tartaric acid, and (−)-di-p-toluoyl-L-tartaric acid; and
(ii) subjecting the compound IV obtained in the step (i) to saponification by treating it with a base or a buffer to obtain the(S)-enantiomer of the compound III, the compound IIIa.

In an embodiment, the solvent used in step (i) of the above process is selected from the solvents described in one or more of the embodiments set forth herein above in respect of the resolution of the racemic mixture of the compound III.

In an embodiment, in the step (i), the racemic mixture of the compound III is treated with the chiral acid at a temperature from about 20° C. to about 70° C.

In an embodiment, in the step (i), the reaction mixture may be stirred for a suitable time. The stirring time may range from about 30 minutes to about 10 hours, or longer.

In an embodiment, in the step (ii), saponification of the compound IV is carried out in the presence of a base.

The base may be selected from an inorganic base or an organic base.

The inorganic base may be selected from an alkali metal or an alkaline earth metal carbonates or bicarbonates such as sodium carbonate, potassium carbonate, sodium bicarbonate, potassium bicarbonate; alkali metal or alkaline earth metal hydroxides such as sodium hydroxide, potassium hydroxide, calcium hydroxide, lithium hydroxide; or an alkali metal alkoxide such as sodium methoxide, potassium methoxide or the like.

The organic base may be selected from triethylamine, diisopropylethylamine, N,N-dimethylaniline, 4-bromo-N,N-dimethylaniline, pyridine, 2-bromopyridine, 3-bromopyridine, 4-bromopyridine, 4-dimethylaminopyridine, di-tertbutyl pyridine, 2,6-di-tert-butyl-4-methylpyridine, quinoline, tri-n-butylamine, N-methylmorpholine, 2,6-lutidine, imidazole, 1,5-diazabicyclo[4.3.0]non-5-ene, 1,8-diazabicyclo[5.4.0]undec-7-ene, 1,4-diazabicyclo[2.2.2]octane (DABCO), and the like.

In an embodiment, in the step (ii), the saponification of the compound IV is carried out in the presence of a buffer.

As used herein, the term “buffer” refers to mixture of a weak acid and its conjugate base or a mixture of a weak base and its conjugate acid, wherein the addition of an acid or a base does not change the pH value of the mixture.

In an embodiment, the buffer is selected from potassium dihydrogen phosphate, dipotassium hydrogen phosphate, tripotassium phosphate, sodium dihydrogen phosphate, disodium hydrogen phosphate, tri-sodium phosphate, or hydrates thereof. In an embodiment, a mixture of one or more of the buffers may be used.

In an embodiment, the buffer is selected from tri-sodium phosphate or its dodecahydrate.

In an embodiment, the step (ii) is carried out in the presence of a solvent selected from water, an organic solvent, or a mixture thereof, wherein the organic solvent includes, but is not limited to, alcohols such as methanol, ethanol, 1,2-ethanediol, methoxyethanol, isopropanol, n-propanol, butanol and the like; hydrocarbons such as toluene, xylene, chlorobenzene, heptane, hexane, cyclohexane and the like; ethers such as dimethyl ether, diethyl ether, diisopropyl ether, tert-butyl methyl ether, dibutyl ether, dimethoxyethane, diethoxyethane, tetrahydrofuran, dioxane and the like; halogenated hydrocarbons such as dichloromethane, dichloroethane, chloroform, ethylene dichloride, and the like; esters such as ethyl acetate, isopropyl acetate, isobutyl acetate, t-butyl acetate and the like; nitriles such as acetonitrile, propionitrile, butyronitrile, benzonitrile and the like; ketone such as acetone, methyl isobutyl ketone, methyl ethyl ketone, cyclohexanone and the like; polar solvent such as dimethylformamide, dimethyl sulfoxide, dimethyl acetamide, or a mixture thereof.

In an embodiment, the step (ii) is carried out in the presence of a solvent selected from water, an organic solvent or a mixture thereof; wherein the organic solvent is selected from methanol, ethanol, 1,2-ethanediol, methoxyethanol, isopropanol, n-propanol, ethyl acetate, isopropyl acetate, acetone, methyl ethyl ketone, cyclohexanone, toluene, xylene, acetonitrile, dimethylformamide, or dimethyl sulfoxide.

In an embodiment, in the step (ii), the saponification of the compound IV is carried out at a temperature ranging from about 20° C. to about 60° C.

In an embodiment, in the step (ii), the reaction mixture may be stirred for a suitable time. The stirring time may range from about 30 minutes to about 10 hours, or longer.

In an embodiment, in the step (ii), the pH of the reaction mass was adjusted between 7.0 to 7.5.

In an embodiment, the compound IIIa is isolated by filtration, centrifugation or by any other method known in the art.

In an embodiment, the present invention provides a process for the preparation of the compound IVa, which is a diastereomeric salt of 4-(4-cyano-2-methoxyphenyl)-5-ethoxy-2,8-dimethyl-1,4-dihydro-1,6-naphthyridine-3-carboxylic acid (the compound III) with (+)-dibenzoyl-D-tartaric acid.

The process for the preparation of the compound IVa represented by the following formula;

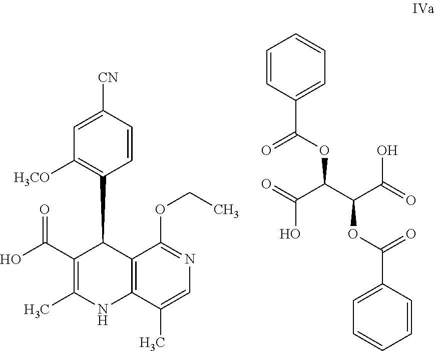 [SMILES: CCOc1ncc(C)c2c1[C@H](c1ccc(C#N)cc1OC)C(C(=O)O)=C(C)N2 ; O=C(O[C@H](C(=O)O)[C@H](OC(=O)c1ccccc1)C(=O)O)c1ccccc1]

comprises treating a racemic mixture of the compound III with (+)-dibenzoyl-D-tartaric acid represented by the compound of formula V (the “compound V”);

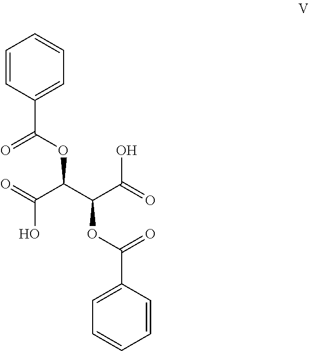 [SMILES: O=C(O[C@H](C(=O)O)[C@H](OC(=O)c1ccccc1)C(=O)O)c1ccccc1]

to obtain the diastereomeric salt, the compound IVa.

The racemic mixture of the compound III as used herein in the process of the present invention is prepared as described herein in examples 3 and 11.

In an embodiment, in the process for the preparation of the compound IVa, the racemic mixture i.e. the compound III is treated with (+)-dibenzoyl-D-tartaric acid in the presence of a solvent as described in one or more embodiments set forth herein above.

In another embodiment, in the process for the preparation of the compound IVa, the racemic mixture of compound III is treated with (+)-dibenzoyl-D-tartaric acid in the presence of a solvent selected from methanol, ethanol, 1,2-ethanediol, methoxyethanol, isopropanol, n-propanol, butanol, tetrahydrofuran (THF), dimethyltetrahydrofuran, dioxane, ethyl acetate, isopropyl acetate, isobutyl acetate, t-butyl acetate, water or a mixture thereof.

In an embodiment, in the process of the preparation of the compound IVa, the racemic mixture of compound III is treated with (+)-dibenzoyl-D-tartaric acid in the presence of a mixture of solvents comprising of water and an organic solvent selected from methanol, ethanol, 1,2-ethanediol, methoxyethanol, isopropanol, n-propanol, butanol, tetrahydrofuran (THF), dimethyltetrahydrofuran, dioxane, ethyl acetate, isopropyl acetate, isobutyl acetate, or t-butyl acetate or a mixture thereof.

In an embodiment, in the process of the preparation of the compound IVa, the racemic mixture of compound III is treated with (+)-dibenzoyl-D-tartaric acid in the presence a mixture of solvents comprising water and an alcohol selected from methanol, ethanol, 1,2-ethanediol, methoxyethanol, isopropanol, n-propanol, or butanol.

In one embodiment, in the process for the preparation of the compound IVa, the racemic mixture of the compound III is treated with (+)-dibenzoyl-D-tartaric acid at a temperature ranging from about 20° C. to about 70° C.

In an embodiment, in the process for the preparation of the compound IVa, the reaction mixture may be stirred for a suitable time. The stirring time may range from about 30 minutes to about 10 hours, or longer.

In an embodiment, the chiral purity of the compound IIIa obtained by the process of the present invention is >90%.

In an embodiment, the chiral purity of the compound IIIa obtained by the process of the present invention is >95%.

In an embodiment, the chiral purity of the compound IIIa obtained by the process of the present invention is >97%.

The chiral purity of the compounds obtained by the process of the present invention can be determined using high performance liquid chromatography (HPLC) under conditions that are generally known to those skilled in the art.

In one further aspect, the present invention provides a process for the preparation of finerenone, the compound I,

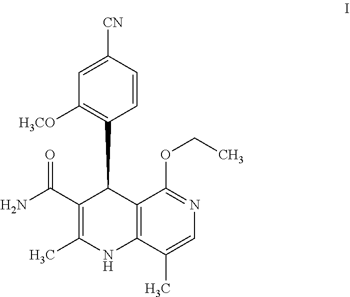 [SMILES: CCOc1ncc(C)c2c1[C@H](c1ccc(C#N)cc1OC)C(C(N)=O)=C(C)N2]

comprising the steps of;
a) obtaining a diastereomeric salt of 4-(4-cyano-2-methoxyphenyl)-5-ethoxy-2,8-dimethyl-1,4-dihydro-1,6-naphthyridine-3-carboxylic acid with a chiral acid represented by the compound of formula IV (the “compound IV”);

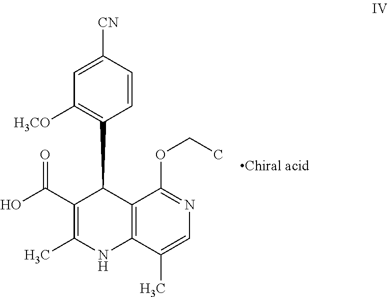 [SMILES: CCOc1ncc(C)c2c1[C@H](c1ccc(C#N)cc1OC)C(C(=O)O)=C(C)N2]

wherein the chiral acid is selected from the group consisting of L-(+) tartaric acid, D-(−) tartaric acid, (+)-dibenzoyl-D-tartaric acid, (−)-dibenzoyl-L-tartaric acid, (+)-di-p-toluoyl-D-tartaric acid, and (−)-di-p-toluoyl-L-tartaric acid;
b) subjecting the compound IV obtained in step (a) to saponification to obtain the(S)-enantiomer of 4-(4-cyano-2-methoxyphenyl)-5-ethoxy-2,8-dimethyl-1,4-dihydro-1,6-naphthyridine-3-carboxylic acid represented by the compound of formula IIIa (the “compound IIIa”);

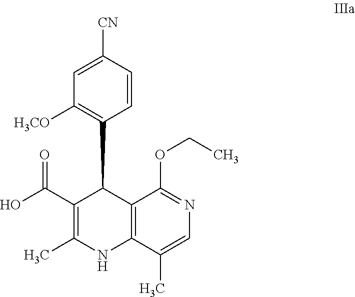 [SMILES: CCOc1ncc(C)c2c1[C@H](c1ccc(C#N)cc1OC)C(C(=O)O)=C(C)N2]

c) converting the compound IIIa obtained in the step (b) to finerenone, the compound I; and
d) optionally, purifying the compound I.

In an embodiment, in the process for the preparation of finerenone (the compound I), the step (a) for obtaining compound IV is carried out by treating the racemic mixture of compound III with a chiral acid selected from the group consisting of L-(+) tartaric acid, D-(−) tartaric acid, (+)-dibenzoyl-D-tartaric acid, (−)-dibenzoyl-L-tartaric acid, (+)-di-p-toluoyl-D-tartaric acid, and (−)-di-p-toluoyl-L-tartaric acid; in the presence of a solvent, wherein the solvent may be selected from those described in one or more embodiments set forth herein above.

In an embodiment, in the process for the preparation of finerenone (the compound I), the chiral acid used in the step (a) is (+)-dibenzoyl-D-tartaric acid.

Accordingly, in an embodiment, the present invention provides a process for the preparation of finerenone (the compound I);

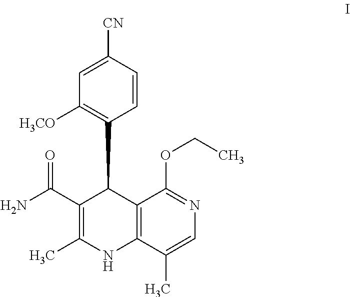 [SMILES: CCOc1ncc(C)c2c1[C@H](c1ccc(C#N)cc1OC)C(C(N)=O)=C(C)N2]

comprising the steps of;
a-1) obtaining a diastereomeric salt of 4-(4-cyano-2-methoxyphenyl)-5-ethoxy-2,8-dimethyl-1,4-dihydro-1,6-naphthyridine-3-carboxylic acid with (+)-dibenzoyl-D-tartaric acid represented by the compound of formula IVa (the “compound IVa”);

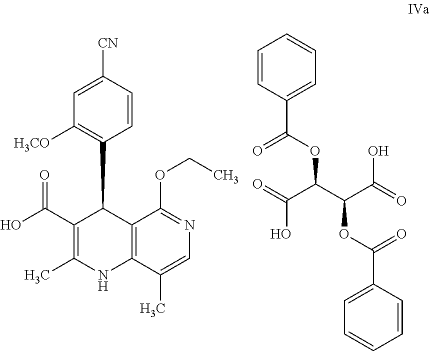

b-1) subjecting the compound IVa obtained in the step (a-1) to saponification to obtain the(S)-enantiomer of 4-(4-cyano-2-methoxyphenyl)-5-ethoxy-2,8-dimethyl-1,4-dihydro-1,6-naphthyridine-3-carboxylic acid represented by the compound of formula IIIa (the “compound IIIa”);

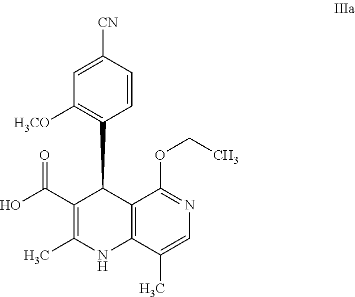 [SMILES: CCOc1ncc(C)c2c1[C@H](c1ccc(C#N)cc1OC)C(C(=O)O)=C(C)N2]

c-1) converting the compound IIIa obtained in the step (b-1) to finerenone, the compound I; and
d-1) optionally, purifying the compound I.

In an embodiment, in the process for the preparation of finerenone (the compound I), the step (a-1) for obtaining compound IVa is carried out by treating a racemic mixture of the compound III with (+)-dibenzoyl-D-tartaric acid in the presence of a solvents, wherein the solvent may be selected from those described in one or more embodiments set forth herein above.

In a further embodiment, in the process for the preparation of finerenone (the compound I), the solvent used in the step (a-1) is selected from methanol, ethanol, 1,2-ethanediol, methoxyethanol, isopropanol, n-propanol, butanol, tetrahydrofuran (THF), dimethyl-tetrahydrofuran, dioxane, acetone, ethyl acetate, isopropyl acetate, isobutyl acetate, t-butyl acetate, water or a mixture thereof.

In a further embodiment, in the process for the preparation of finerenone (the compound I), the step (a-1) is carried out in a mixture of solvents comprising of water and an organic solvent selected from methanol, ethanol, 1,2-ethanediol, methoxyethanol, isopropanol, n-propanol, butanol, tetrahydrofuran (THF), dimethyltetrahydrofuran, dioxane, acetone, ethyl acetate, isopropyl acetate, isobutyl acetate, or t-butyl acetate.

In an embodiment, in the process for the preparation of finerenone (the compound I), the step (a-1) is carried out in a mixture of solvents comprising water and an alcohol selected from methanol, ethanol, 1,2-ethanediol, methoxyethanol, isopropanol, n-propanol, or butanol.

In one embodiment, in the process for the preparation of finerenone (the compound I), the step (a-1) is carried out at a temperature ranging from about 20° C. to about 70° C.

In a further embodiment, in the process for the preparation of finerenone (the compound I), the step (a-1) is carried out at a temperature ranging from about 50° C. to about 70° C.

In an embodiment, in the process for the preparation of finerenone (the compound I), the saponification of the diastereomeric salt i.e. the compound IVa in the step (b-1) is carried out in presence of a base or a buffer.

The term “saponification” as used herein refers to the hydrolysis of diastereomeric salts i.e. the compound IV or the compound IVa using a base or a buffer.

In an embodiment, in the process for the preparation of finerenone (the compound I), the saponification of the diastereomeric salt i.e. the compound IVa in the step (b-1) is carried out in presence of a base selected from the bases as described herein above in one or more of the embodiments.

In an embodiment, in the process for the preparation of finerenone (the compound I), the saponification of the diastereomeric salt i.e. the compound IVa in the step (b-1) is carried out in presence of a buffer selected from the buffer as described herein above in one or more of the embodiments.

In an embodiment, the step (b-1) is carried out in the presence of a solvent as described herein above in one or more of the embodiments.

In an embodiment, in the step (b-1), saponification of the compound IVa is carried out at a temperature ranging from about 20° C. to about 60° C.

In an embodiment, in the step (b-1), the reaction mixture may be stirred for a suitable time. The stirring time may range from about 30 minutes to about 10 hours, or longer.

In an embodiment, in the step (b-1), the pH of the reaction mass was adjusted between 7.0 to 7.5.

In an embodiment, in the process for the preparation of finerenone (the compound I), the step (c-1) involves reacting the compound IIIa with 1,1-carbodiimidazole (CDI) and 4-(dimethylamino) pyridine (DMAP) in a suitable solvent such as dimethylformamide (DMF), followed by addition of aqueous ammonia, and further workup to obtain Finerenone (the compound I).

In a further embodiment, in the process for the preparation of finerenone (the compound I), the step (d-1) involves purification of finerenone (the compound I) by recrystallizing the compound I obtained in the step (c-1) in an alcoholic solvent selected from methanol, ethanol, industrial solvent, 1,2-ethanediol, methoxyethanol, isopropanol, n-propanol, or butanol.

In an embodiment, in the process for the preparation of finerenone (the compound I), the step (d-1) involves purification of finerenone (the compound I) by recrystallizing the compound I obtained in the step (c-1) in a mixture of solvents comprising water and an organic solvent selected from methanol, ethanol, industrial solvent, 1,2-ethanediol, methoxyethanol, isopropanol, n-propanol, or butanol.

In an embodiment, finerenone (the compound I) obtained by the process of the present invention is isolated by filtration, centrifugation or by a method known in the art.

In an embodiment, the present invention provides diastereomeric salts of 4-(4-cyano-2-methoxyphenyl)-5-ethoxy-2,8-dimethyl-1,4-dihydro-1,6-naphthyridine-3-carboxylic acid with dibenzoyl-D-tartaric acid. The diastereomeric salts are represented by the compound of formula IVa (the “compound IVa”), the compound of formula IVb (the “compound IVb”), the compound of formula IVc (the “compound IVc”), and the compound of formula IVd (the “compound IVd”);

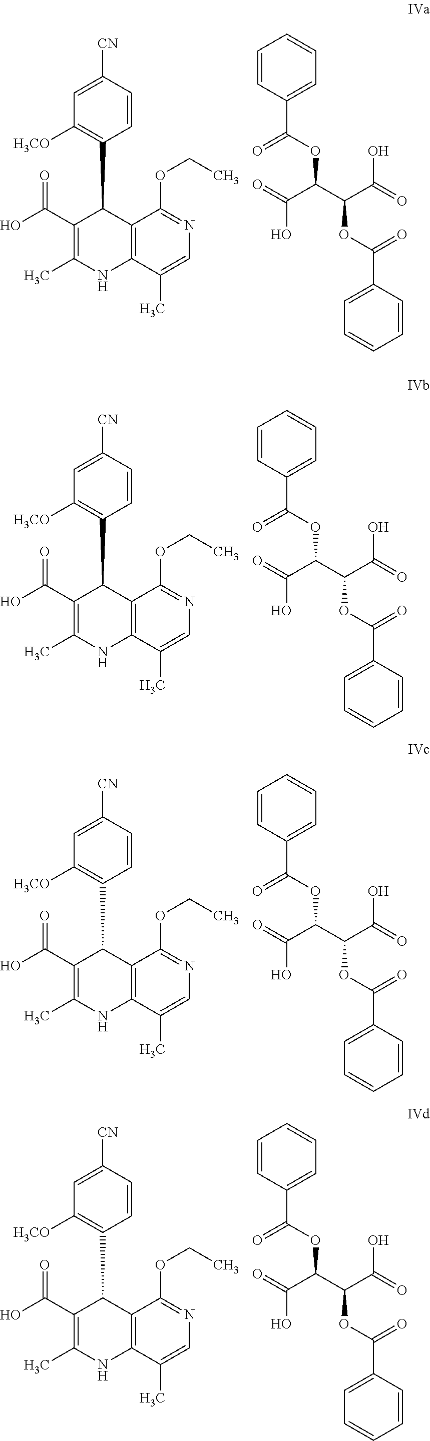

In a specific embodiment, the present invention relates to a diastereomeric salt represented by the compound of formula IVa (the “compound IVa”),

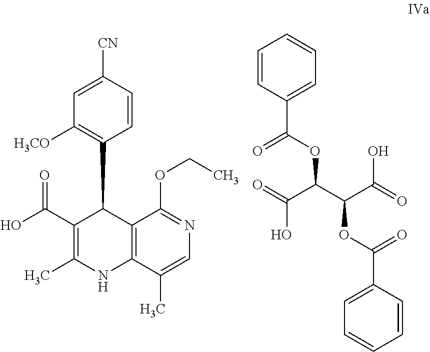

In another embodiment, the present invention relates to use of the compound IVa in the preparation of finerenone (the compound I).

In an embodiment, the use of the compound IVa in the preparation of finerenone can be carried out by the process(es) as described herein above.

In an embodiment, finerenone (the compound I) obtained by the above described process may be recrystallized from a solvent which includes, but is not limited to, alcohols such as methanol, ethanol, industrial solvent, isopropanol (IPA), n-propanol, n-butanol and the like; organic acids such as acetic acid, formic acid, lactic acid and the like; esters such as methyl acetate, ethyl acetate, n-propyl acetate, isopropyl acetate, isobutyl acetate, t-butyl acetate and the like; hydrocarbons such as methylene dichloride, ethylene chloride, toluene, xylene, heptane, hexane, cyclohexane and the like; nitriles such as acetonitrile, propionitrile and the like; ethers such as diethyl ether, diisopropyl ether, methyl tert-butyl ether, tetrahydrofuran and the like; ketones such as acetone, methyl ethyl ketone, ethyl methyl ketone, methyl isobutyl ketone and the like; water; and a mixture thereof.

In an embodiment, finerenone (the compound I) obtained by the process of the present invention has chiral purity of greater than 95%.

In an embodiment, finerenone (the compound I) obtained by the process of the present invention has chiral purity of greater than 97%.

In an embodiment, finerenone (the compound I) obtained by the process of the present invention has chiral purity of greater than 99%.

In an embodiment, finerenone (the compound I) obtained by the process of the present invention has chiral purity of greater than 99.9%.

In an embodiment, finerenone (the compound I) obtained by the process of the present invention has 100% chiral purity.

In an embodiment, finerenone (the compound I) obtained by the process of the present invention has a purity of ≥99.0%; wherein the level of the impurities designated herein as impurity A, impurity B, impurity C, or impurity D represented by the following structural formulae is less than 0.15% w/w relative to the amount of finerenone.

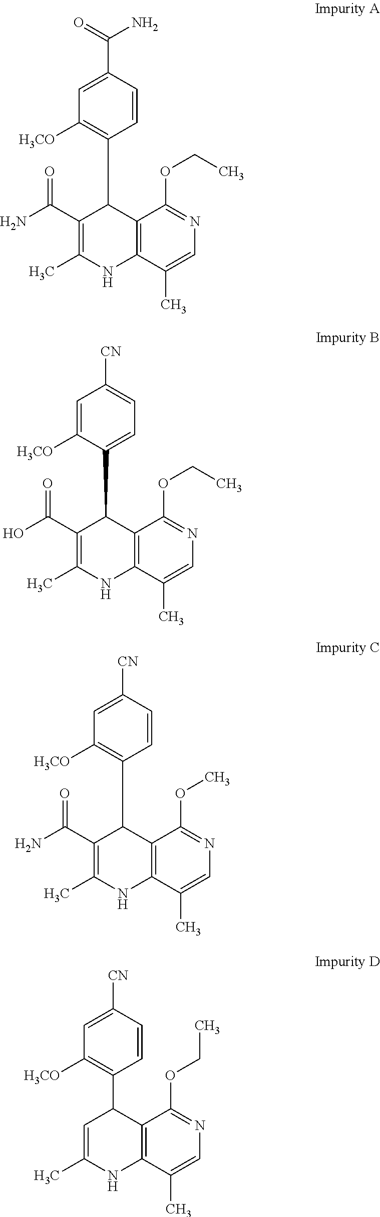

The level of impurities in finerenone (the compound I) is determined by High Performance Liquid Chromatography (HPLC).

In an embodiment, finerenone (the compound I) is obtained in a purity of ≥99.0%, and wherein the level any of the impurities as described above is less than 0.10%.

In an embodiment, finerenone (the compound I) is obtained in a purity of ≥99.0%, and wherein the level any of the impurity as described above is less than 0.05%.

In an embodiment, finerenone (the compound I) is obtained in a purity of ≥99.0%, and wherein the level any of the impurity as described above is not detected.

In an embodiment, finerenone (the compound I) is obtained in a purity of ≥99.8% and wherein the level of the impurity as described above is less than 0.15%.

The impurities set forth above can be detected by standard analytical techniques known to a person of skill in the art such as High Performance Liquid Chromatography (HPLC), Mass Spectrometry (MS) and the like.

In an aspect, the present invention provides a pharmaceutical composition comprising finerenone (the compound I) obtained by the process of the present invention as herein described, and at least one pharmaceutically acceptable excipient. The pharmaceutically acceptable excipient may be suitably selected by a person of skill in the art depending on the dosage form and the route of administration.

In an embodiment, the present invention provides finerenone (the compound I) obtained by the process of the present invention, having a D₁₀ particle size of less than about 100 microns, preferably less than about 50 microns, more preferably less than about 30 microns.

In one embodiment, the present invention provides finerenone (the compound I) obtained by the process of the present invention, having a D₅₀ particle size of less than about 150 microns, preferably less than about 100 microns, more preferably less than about 60 microns.

In one embodiment, the present invention provides finerenone (the compound I) obtained by the process of the present invention, having a D₉₀ particle size of less than about 250 microns, preferably less than about 150 microns, more preferably less than about 100 microns.

The particle size for finerenone (the compound I) disclosed herein above can be obtained by, for example, any milling, grinding, micronizing or other particle size reduction method known in the art to bring the solid state finerenone into any of the foregoing desired particle size range.

While the present invention has been described in terms of its specific embodiments, certain modifications and equivalents will be apparent to those skilled in the art and are intended to be included within the scope of the present invention.

The examples that follow are provided to enable one skilled in the art to practice the invention and are merely illustrative of the invention. The examples should not be read as limiting the scope of the invention as defined in the features and advantages.

### EXAMPLES

### General Methods:

### HPLC Method (Instrumental Settings):

High performance liquid chromatography (HPLC) was performed with the conditions described below for determining purity:

### A] for Chiral Purity:

### Reagents and Solvents:

n-Hexane (Rankem Grade); 2-Propanol (Rankem Grade); Methanol (Rankem Grade); 1-Propanol (Rankem Grade)

### Chromatographic Conditions:

Apparatus: A High Performance Liquid Chromatograph equipped with quaternary gradient pumps, variable wavelength UV detector attached with data recorder and integrator software.
Column: Chiralpak AS-H, 250×4.6 mm, 5μ
Column temperature: 40° C.
Mobile Phase: n-Hexane:2-Propanol:1-Propanol:Diethylamine (60:30:10:0.1, v/v
Diluent: Methanol:Mobile phase (50:50 V/V)
Flow Rate: 1.2 mL/minute
Detection: UV 252 nm
Injection Volume: 20 μL

The retention time of finerenone R-isomer is about 10.0 minutes and the retention time of finerenone is about 25.0 minutes under these conditions.

### B] for Chemical Purity:

### Reagents and Solvents:

Water (Milli Q or equivalent); Acetonitrile (Gradient, Advent) Orthophosphoric acid (AR Grade, Merck)
Dipotassium hydrogen phosphate anhydrous (AR grade, Rankem)

### Chromatographic Conditions:

Apparatus: A High Performance Liquid Chromatograph equipped with quaternary gradient pumps, variable wavelength UV detector attached with data recorder and integrator software or equivalent.
Column: Waters XBridge C8, 150×4.6 mm, 3.5μ
Column temperature: 45° C.
Mobile Phase: Mobile Phase A=Buffer:Acetonitrile (95:05, v/v)
Buffer: 0.01M Dipotassium hydrogen phosphate in water adjust pH 7.30 with diluted orthophosphoric acid

Mobile Phase B = Acetonitrile (100%)

| Time (min.) | % Mobile Phase A | % Mobile Phase B |
|---|---|---|
| 0.01 | 90 | 10 |
| 30 | 55 | 45 |
| 40 | 30 | 70 |
| 48 | 30 | 70 |
| 49 | 90 | 10 |
| 55 | 90 | 10 |

Diluent-1: Acetonitrile:Water (40:60, v/v)
Diluent-2: Acetonitrile:Water (80:20, v/v)
Flow Rate: 1.0 mL/minute
Detection: UV 252 nm
Injection Volume: 10 μL The retention time of finerenone is about 17.5 minutes under these conditions.

### Examples

### Example 1: Preparation of 2-cyanoethyl 2-(4-cyano-2-methoxybenzylidene)-3-oxobutanoate (Compound VIII)

4-formyl-3-methoxybenzonitrile (50 gm) and 2-cyanoethylacetoacetate (50.35 gm) were added in ethanol (200 ml) followed by adding acetic acid (1.86 gm) and piperidine (2.64 gm) into the reaction mass at a temperature 20° C.-30° C. The reaction mixture was stirred for about 5-6 hours at the temperature of about 25° C. to 30° C. After completion of the reaction, ethanol (100 ml) was added, and the reaction mass was stirred at a temperature of 25° C. to 30° C. for about 2 hours. The reaction mass was filtered, washed with ethanol, and the resulting solid was dried in Vacuum Tray Drier (VTD) at a temperature of about 50° C. to 55° C. to obtain 2-cyanoethyl 2-(4-cyano-2-methoxybenzylidene)-3-oxobutanoate (Compound VIII). Yield: 83 gm (89%); HPLC purity: 97.37%

### Example 2: Preparation of 2-cyanoethyl 4-(4-cyano-2-methoxyphenyl)-2,8-dimethyl-5-oxo-1,4,5,6-tetrahydro-1,6-naphthyridine-3-carboxylate (Compound VII)

Compound VIII (70 gm) and 4-amino-5-methylpyridin-2 (1H)-one (26.01 gm) were added in ethanol (420 ml) at a temperature of about 20° C. to 30° C., and further refluxed at a temperature of about 75° C. to about 80° C. for about 25-27 hours. After completion of the reaction, the reaction mixture was cooled to a temperature of about 25° C. to 30° C. and stirred for about 2 hours. The reaction mass was filtered, washed with ethanol, and the resulting solid was dried in Vacuum Tray Drier (VTD) at a temperature of about 50° C. to 55° C. to obtain 2-cyanoethyl 4-(4-cyano-2-methoxyphenyl)-2,8-dimethyl-5-oxo-1,4,5,6-tetrahydro-1,6-naphthyridine-3-carboxylate (Compound VII). Yield: 64 gm (76.0%); HPLC purity: 99.7%

### Example 3: Preparation of Racemic Mixture of 4-(4-cyano-2-methoxyphenyl)-5-ethoxy-2,8-dimethyl-1,4-dihydro-1,6-naphthyridine-3-carboxylic acid (Compound III)

Compound VII (10 gm) and triethyl orthoformate (12.83 gm) were charged in dimethylformamide (DMF) (40 ml), followed by adding sulphuric acid (0.19 gm), and the reaction mass was heated to a temperature of about 85° C. to 90° C. for about 9-12 hours to obtain 2-cyanoethyl 4-(4-cyano-2-methoxyphenyl)-5-ethoxy-2,8-dimethyl-1,4-dihydro-1,6-naphthyridine-3-carboxylate (Compound VI) which is then cooled to a temperature of about 25-30° C. Meanwhile, sodium hydroxide (NaOH) (4.45 gm) in water (20 ml) was added in another 4 neck flask, stirred for about 15-30 minutes, and then the compound VI was added at a temperature of about 20° C. to 30° C. to it, and further, the reaction mass was stirred for about 1-2 hours. After completion of the reaction, water was added, pH of reaction mass was adjusted between 6.5 to 7.5 using HCl and the reaction mass was stirred for about 2 hours. The reaction mass was filtered, washed and the obtained solid was dried in Air Tray Drier (ATD) at a temperature of about 50° C. to about 55° C. to obtain crude title compound (8.25 gm). The crude compound was purified by using a mixture of acetone (33 ml) and water (8.25 ml) to obtain pure racemic mixture of 4-(4-cyano-2-methoxyphenyl)-5-ethoxy-2,8-dimethyl-1,4-dihydro-1,6-naphthyridine-3-carboxylic acid (Compound III). Yield: 6.74 gm (72.0%); HPLC purity: 99.87%

### Example 4: Preparation of Dibenzoyltartaric Acid Salt of 4-(4-cyano-2-methoxyphenyl)-5-ethoxy-2,8-dimethyl-1,4-dihydro-1,6-naphthyridine-3-carboxylic acid (Compound IVa)

Compound III (15 gm) was charged in a mixture of methanol: water (3:1, 42 ml), then (+)-dibenzoyl-D-tartaric acid (D-(+)-DBTA, 7.08 gm) was added. The reaction mass was heated to a temperature of about 60° C. to 65° C. for about 2 hours, cooled to a temperature of about 20-30° C., and the reaction mass was stirred for about 2 hours. The reaction mass was filtered, washed with a mixture of methanol: water (3:1, 6 ml), the resulting solid was dried in Vacuum Tray Drier (VTD) at a temperature of about 55-60° C. to obtain dibenzoyltartaric acid (DBTA) salt of 4-(4-cyano-2-methoxyphenyl)-5-ethoxy-2,8-dimethyl-1,4-dihydro-1,6-naphthyridine-3-carboxylic acid (Compound IVa). Yield: 13.34 gm (91.47%); HPLC purity: 73.67%.

### Example 5: Preparation of (S)-Enantiomer of 4-(4-cyano-2-methoxyphenyl)-5-ethoxy-2,8-dimethyl-1,4-dihydro-1,6-naphthyridine-3-carboxylic acid (Compound IIIa)

Compound IVa (3 gm) was charged in a mixture of ethanol and water (2:8, 130 ml), stirred for about 15 minutes. pH of the reaction mass was adjusted between 7.0 to 7.5 by using 10% aq. trisodium phosphate solution. The reaction mass was stirred for about 15 minutes, heated at the temperature of about 50° C. to 55° C. for about 30 minutes, cooled at a temperature of about 20-30° C., and then further stirred for about 2 hours. The reaction mass was filtered, washed with a mixture of ethanol and water (2:8, 26 ml). The resulting solid was dried in Vacuum Tray Drier (VTD) at a temperature of about 55-60° C. to obtain the(S)-enantiomer of 4-(4-cyano-2-methoxyphenyl)-5-ethoxy-2,8-dimethyl-1,4-dihydro-1,6-naphthyridine-3-carboxylic acid (Compound IIIa).

Yield: 6.0 gm (90.50%); HPLC purity: 96.29% Chiral purity S-isomer 90.62% and R-Isomer 9.38%.

### Example 6: Preparation of Finerenone (Compound I)

Compound IIIa (4 gm), CDI (4.76 gm) and dimethylaminopyridine (DMAP) (0.13 gm) were added in DMF (8.0 ml), and the reaction mass was stirred for about 2-3 hrs at a temperature of about 20° C. to 30° C. to obtain 4 (S)-[5-ethoxy-3-(1H-imidazole-1-carbonyl)-2,8-dimethyl-1,4-dihydro-1,6-naphthyridin-4-yl]-3-methoxy-benzonitrile (Compound II). Aq. ammonia (16 ml) was added to the reaction mass, and heated to a temperature of about 80-90° C. for about 2-3 hours. After completion of the reaction, the reaction mass was cooled to the temperature of about 20-30° C., stirred for about 15 minutes to obtain compound I. Meanwhile, water (64 ml) was added in another 4 neck flask, then the compound was added to it at a temperature of about 20° C. to 30° C., and further the reaction mass was stirred for about 15-20 minutes, sodium acetate (8 gm) was added, pH of reaction mass was adjusted between 6.5 to 7.5 using HCl and the reaction mass was stirred for about 2 hours. The reaction mass was filtered, washed and the resulting solid was dried in Air Tray Drier (ATD) at about 50-55° C. to obtain crude title compound (compound I, 3.12 gm). The crude compound I was purified in a mixture of ethyl acetate and methanol. Chiral purity: R-isomer 9.42, and S-isomer 90.58%.

### Example 7: Purification of Finerenone (Compound I)

Finerenone, the compound I (0.8 gm) was charged in ethanol (4 ml) at a temperature of about 20 to 30° C., stirred, and then the temperature of the reaction mass was raised to about 60-70° C., stirred for about 1 hour, and then cooled to the temperature of about 20-30° C. The reaction mass was further stirred for about 1 hour. The reaction mass was filtered, washed with ethanol, obtained solid was dried in Vacuum Tray Drier (VTD) to obtain pure finerenone, the compound I. Yield: 0.55 gm (62.50%)

### Example 8: Preparation of 2-cyanoethyl-4-(4-cyano-2-methoxyphenyl)-2,8-dimethyl-5-oxo-1,4,5,6-tetrahydro-1,6-naphthyridine-3-carboxylate (Compound VII)

2-cyanoethyl 2-(4-cyano-2-methoxybenzylidene)-3-oxobutanoate (compound VIII, 267 gm) and 4-amino-5-methylpyridin-2 (1H)-one (100 gm) was added in an industrial solvent (1000 ml) at a temperature of about 25° C. to 35° C., and then refluxed at a temperature of about 75° C. to about 80° C. for about 27-30 hours. After completion of reaction, the reaction mixture was cooled to a temperature of about 25° C. to about 30°, and stirred for about 2 hours. The reaction mass was filtered, washed with industrial solvent (200 ml) and the resulting solid was dried in Vacuum Tray Dryer (VTD) at a temperature of about 65-75° C. to obtain the title compound VII. Yield: 532 gm (81.75%); HPLC purity: 98.14%

### Example 9: Preparation of 2-cyanoethyl-4-(4-cyano-2-methoxyphenyl)-2,8-dimethyl-5-oxo-1,4,5,6-tetrahydro-1,6-naphthyridine-3-carboxylate (Compound VII)

2-cyanoethyl-2-(4-cyano-2-methoxybenzylidene)-3-oxobutanoate (compound VIII, 66.68 gm) and 4-amino-5-methylpyridin-2 (1H)-one (25 gm) was added to n-butanol (250 ml) at a temperature of about 20° C. to 30° C., and then refluxed at a temperature of about 105° C. and 120° C. for about 25-27 hours. After completion of the reaction, the reaction mixture was cooled to the temperature of about 20-30° C. and stirred for about 2 hours. The reaction mass was filtered, washed with n-butanol (50 ml) and the resulting solid was dried in Vacuum Tray Dryer (VTD) at about 65-75° C. to obtain the title compound VII. Yield: 67 gm (82.28%); HPLC purity: 96.92%.

### Example 10: Preparation of 2-cyanoethyl-4-(4-cyano-2-methoxyphenyl)-2,8-dimethyl-5-oxo-1,4,5,6-tetrahydro-1,6-naphthyridine-3-carboxylate (Compound VII)

2-cyanoethyl 2-(4-cyano-2-methoxybenzylidene)-3-oxobutanoate (compound VIII, 53.34 gm) in 4 lots and 4-amino-5-methylpyridin-2 (1H)-one (20 gm) were added in dimethylformamide (DMF) (210 ml) at a temperature of about 20 to 30° C., and the temperature was raised to about 90-105° C. for about 9-10 hours. After completion of the reaction, the reaction mixture was cooled to a temperature of about 20-30° C. and added water (264 ml), stirred for about 2 hours. The reaction mass was filtered, washed with water (40 ml), and the resulting solid was dried in Air Tray Dryer (ATD) at a temperature of about 55-60° C. to obtain the title compound VII. Yield: 59 gm (90.56%); HPLC purity: 97.35%.

### Example 11: Preparation of Racemic Mixture of 4-(4-cyano-2-methoxyphenyl)-5-ethoxy-2,8-dimethyl-1,4-dihydro-1,6-naphthyridine-3-carboxylic acid (Compound III)

2-cyanoethyl-4-(4-cyano-2-methoxyphenyl)-2,8-dimethyl-5-oxo-1,4,5,6-tetrahydro-1,6-naphthyridine-3-carboxylate (compound VII, 100 gm) and triethyl orthoformate (87.95 gm) were charged in dimethylformamide (400 ml) and then sulphuric acid (7.27 gm) was added, the reaction mass was heated to a temperature of about 85-95° C. for about 9-12 hours to obtain 2-cyanoethyl 4-(4-cyano-2-methoxyphenyl)-5-ethoxy-2,8-dimethyl-1,4-dihydro-1,6-naphthyridine-3-carboxylate (Compound VI), which was then cooled to a temperature of about 25-30° C. Meanwhile, sodium hydroxide (64.29 gm) in water (200 ml) was added in another 4 neck flask, stirred for about 15-30 minutes, and then the compound VI was added at a temperature of about 10-20° C. to it, and further stirred the reaction mass for about 1-2 hours. After completion of the reaction, water (550 ml) and HCl (52 ml) were added, followed by ethyl acetate (150 ml). pH of the reaction mass was adjusted between 5.8 to 6.8 using HCl and the reaction mass was stirred for about 2 hours. The reaction mass was filtered, washed with water (200 ml) followed by ethyl acetate (200 ml), and the obtained solid was dried in Air Tray Dryer (ATD) at the temperature of about 55-65° C. to obtain the title compound III.

Yield: 458.8 gm (97.8%); HPLC purity: 98.22%.

### Example 12: Preparation of Dibenzoyltartaric Acid Salt of 4-(4-cyano-2-methoxyphenyl)-5-ethoxy-2,8-dimethyl-1,4-dihydro-1,6-naphthyridine-3-carboxylic acid (Compound IVa)

Racemic mixture of 4-(4-cyano-2-methoxyphenyl)-5-ethoxy-2,8-dimethyl-1,4-dihydro-1,6-naphthyridine-3-carboxylic acid (compound III, 370 gm) was charged in a mixture of tetrahydrofuran (THF) (1295 ml) and methanol (3125 ml), flushed with methanol (370 ml), followed by stirring the reaction mixture, and then D-(+)-DBTA (164.23 gm) was added and further flushed with methanol (370 ml). The reaction mass was stirred at a temperature of about 25-35° C. for about 10 hours, then the reaction mass was filtered, washed with methanol (370 ml) to obtain wet solid (532 gm), and the obtained wet solid was added into a mixture of THF (925 ml) and methanol (2405 ml) and flushed with methanol (370 ml). The reaction mass was stirred at a temperature of about 25-35° C. for about 3 hours. The reaction mass was filtered, washed with methanol (370 ml) to obtain wet solid, which was dried in Vacuum Tray Drier (VTD) at a temperature of about 45-55° C. to obtain (+)-dibenzoyl-D-tartaric acid salt, the compound IVa. Yield: 335.7 gm (93.37%); HPLC purity: 99.60%; Chiral purity-S-isomer: 97.25% and R-Isomer: 2.75%.

### Example 13: Preparation of Dibenzoyltartaric Acid Salt of 4-(4-cyano-2-methoxyphenyl)-5-ethoxy-2,8-dimethyl-1,4-dihydro-1,6-naphthyridine-3-carboxylic acid (Compound IVa)

Racemic mixture of 4-(4-cyano-2-methoxyphenyl)-5-ethoxy-2,8-dimethyl-1,4-dihydro-1,6-naphthyridine-3-carboxylic acid Compound III (20 gm) was charged in methanol (260 ml), the reaction mass was stirred, and then D-(+)-DBTA (8.88 gm) was added. The reaction mass was stirred to a temperature of about 40° C. for about 6 hours, cooled to a temperature of about 20-30° C., and further stirred the reaction mass for about 10 minutes. The reaction mass was filtered, washed with methanol (20 ml), the resulting solid was dried in Vacuum Tray Drier (VTD) at about 45-55° C. to obtain dibenzoyltartaric acid salt, the title compound IVa. Yield: 18.50 gm (95.16%); HPLC purity: 99.24%, Chiral purity S-isomer: 94.34% and R-Isomer: 5.66%.

### Example 14: Purification of Dibenzoyltartaric Acid Salt of 4-(4-cyano-2-methoxyphenyl)-5-ethoxy-2,8-dimethyl-1,4-dihydro-1,6-naphthyridine-3-carboxylic acid (Compound IVa)

Dibenzoyltartaric acid salt of 4-(4-cyano-2-methoxyphenyl)-5-ethoxy-2,8-dimethyl-1,4-dihydro-1,6-naphthyridine-3-carboxylic acid (compound IVa, 100 gm) was added in tetrahydrofuran (700 ml), and the reaction mass was stirred for about 1-2 hours at a temperature of about 50-60° C. The reaction mass was cooled gradually to a temperature of about 25-35° C., and stirred for about 2 hours. The reaction mass was filtered, washed and the resulting solid was dried in Vacuum Tray Drier (VTD) at a temperature of about 50-60° C. to obtain pure title compound IVa.

Yield: 8.44 gm (87.01%); HPLC Purity: 99.63%. Chiral purity: R-isomer: 3.13, and S-isomer: 96.87%, HPLC Purity: 99.63%.

### Example 15: Preparation of (S)-Enantiomer of 4-(4-cyano-2-methoxyphenyl)-5-ethoxy-2,8-dimethyl-1,4-dihydro-1,6-naphthyridine-3-carboxylic acid (Compound IIIa)

(+)-Dibenzoyl-D-tartaric acid salt of 4S)-4-(4-cyano-2-methoxyphenyl)-5-ethoxy-2,8-dimethyl-1,4-dihydro-1,6-naphthyridine-3-carboxylic acid (compound IVa, 59 gm) was charged in a mixture of water (295 ml) and ethyl acetate (59 ml) (5:1), stirred and cooled the reaction mass at a temperature of 10-20° C. 10% aq. tri-sodium phosphate dodecahydrate solution (42.56 gm trisodium phosphate dodecahydrate dissolved in 413 ml water) was added to the reaction mass at a temperature of about 10-20° C. The reaction mass was stirred for about 5 hours at a temperature of 10-20° C., pH of reaction mass was adjusted between 7.1 to 7.8, and further stirred the reaction mass for about 2 hours, filtered, washed with water (118 ml) followed by toluene (177 ml). The resulting solid was dried in Vacuum Tray Drier (VTD) at a temperature of about 25-35° C. to get the title compound IIIa. Yield: 30 gm (99.70%); HPLC purity: 99.11%. Chiral purity: S-isomer 97.26% and R-Isomer 2.74%.

### Example 16: Preparation of (S)-Enantiomer of 4-(4-cyano-2-methoxyphenyl)-5-ethoxy-2,8-dimethyl-1,4-dihydro-1,6-naphthyridine-3-carboxylic acid (Compound IIIa)

Dibenzoyltartaric acid salt of 4-(4-cyano-2-methoxyphenyl)-5-ethoxy-2,8-dimethyl-1,4-dihydro-1,6-naphthyridine-3-carboxylic acid (compound IVa, 10 gm) was charged in water (80 ml), stirred for about 15-20 minutes at a temperature of 10-20° C. pH of the reaction mass was adjusted between 7.0 to 7.5 by using aq. tri-sodium phosphate solution (7.2 gm tri-sodium phosphate dissolved in 70 ml water). Toluene (30 ml) was added to the reaction mass, stirred for about 3 hours, filtered, washed with toluene. The resulting solid was dried in Air Tray Drier (ATD) at about 45-50° C. to obtain the title compound IIIa. Yield: 4.85 gm (95.10%); HPLC purity: 96.84%; Chiral purity (S-isomer)>95%.

### Example 17: Preparation of (S)-Enantiomer of 4-(4-cyano-2-methoxyphenyl)-5-ethoxy-2,8-dimethyl-1,4-dihydro-1,6-naphthyridine-3-carboxylic acid (Compound IIIa)

Dibenzoyltartaric acid salt of 4-(4-cyano-2-methoxyphenyl)-5-ethoxy-2,8-dimethyl-1,4-dihydro-1,6-naphthyridine-3-carboxylic acid (compound IVa, 10 gm) was charged in water (80 ml), stirred for about 15-20 minutes at a temperature of 10-20° C. pH of the reaction mass was adjusted between 7.0 to 7.5 by using aq. tri-sodium phosphate solution (7.2 gm tri-sodium phosphate dissolved in 70 ml water). The reaction mass was stirred for about 3 hours, filtered, washed with water (20 ml). The resulting solid was dried in Vacuum Tray Drier (VTD) at a temperature of about 45-50° C. to obtain the title compound IIIa. Yield: 4.65 gm (91.18%); HPLC purity: 97.25%; Chiral purity (S-isomer)>95%.

### Example 18: Preparation of Finerenone (Compound I)

(S)-enantiomer of 4-(4-cyano-2-methoxyphenyl)-5-ethoxy-2,8-dimethyl-1,4-dihydro-1,6-naphthyridine-3-carboxylic acid (compound IIIa, 70 gm), 1,1-carbonyldiimidazole (CDI) (70 gm) and dimethylaminopyridine (DMAP) (2.25 gm) were added in dimethylformamide (DMF) (280 ml), the reaction mass was stirred for about 1-2 hours at a temperature of about 25-35° C. to obtain (4S)-4-[5-ethoxy-3-(1H-imidazole-1-carbonyl)-2,8-dimethyl-1,4-dihydro-1,6-naphthyridin-4-yl]-3-methoxybenzonitrile (Compound II). Aq. ammonia (140 ml) was added to the reaction mass, and heated to a temperature of about 80-90° C. for about 2-3 hours. After completion of the reaction, the reaction mass was cooled to a temperature of about 25-35° C., stirred for about 15-30 minutes. Water (210 ml) was added, stirred and then ethyl acetate (70 ml), sodium acetate (35 gm) and water (770 ml) were added to the reaction mass at a temperature of about 25-35° C. pH of reaction mass was adjusted between 6.0 to 7.0 using conc. HCl, and the reaction mass was stirred for about 2 hours. The reaction mass was filtered, washed with water (140 ml) followed by ethyl acetate (70 ml), and the resulting solid was dried in Vacuum Tray Drier (VTD) at a temperature of about 50-60° C. to obtain the crude title compound I (57.98 gm). Crude compound I was purified in ethyl acetate to obtain purified titled compound I.

Yield: 51.20 gm (73.88%)

Chiral purity: R-isomer: 0.14% and S-isomer: 99.86%. HPLC purity: 98.38%.

### Example 19: Preparation of Finerenone (Compound I)

(S)-enantiomer of 4-(4-cyano-2-methoxyphenyl)-5-ethoxy-2,8-dimethyl-1,4-dihydro-1,6-naphthyridine-3-carboxylic acid compound IIIa (10 gm), CDI (10 gm) and dimethylaminopyridine (0.32 gm) were added in dimethylformamide (40 ml), the reaction mass was stirred for about 1-2 hours at the temperature of about 20-30° C. to get 4 (S)-[5-ethoxy-3-(1H-imidazole-1-carbonyl)-2,8-dimethyl-1,4-dihydro-1,6-naphthyridin-4-yl]-3-methoxy-benzonitrile (Compound II). Aq. ammonia (20 ml) was added to the reaction mass and heated to the temperature of about 80-90° C. for about 2-3 hours. After completion of reaction, the reaction mass was cooled to the temperature of about 20-30° C., stirred for about 15-30 minutes. Water (30 ml) was added, stirred and then isopropyl acetate (10 ml), sodium acetate (2.5 gm) and water (130 ml) were added in the reaction mass at the temperature of about 20-30° C., and further stirred the reaction mass for about 15-20 minutes, pH of reaction mass was adjusted between 6.0 to 7.0 using HCl and the reaction mass was stirred for about 2 hours. The reaction mass was filtered, washed with water followed by isopropyl acetate and the resulting solid was dried in Vacuum Tray Drier (VTD) at about 50-55° C. to get crude title compound I (10.54 gm). Crude compound I was purified in isopropyl acetate. Yield: 6.15 gm (81.66%), HPLC purity: 98.17%, Chiral purity: R-isomer: 3.08%, and S-isomer: 96.92%.

### Example 20: Purification of Finerenone (Compound I)

Finerenone, the compound I (42 gm) was charged in industrial solvent (840 ml) at a temperature of about 25° C. to 35° C., stirred, and then the temperature of reaction mass was raised to the temperature of about 75-80° C., stirred for about 1 hour, cooled to about 60-70° C., then filter the reaction mass through suitable filter and washed with industrial solvent (42 ml). Filtrate was collected, and industrial solvent was distilled out up to 7 to 8.5 volume that remains in the reaction mass. Acetic acid (6.72 ml) was added to the reaction mass, temperature was raised to about 75-80° C., stirred for about 1 hour then cooled to a temperature of about 20-30° C. The reaction mass was further stirred for about 2 hours, filtered, washed with industrial solvent, and the obtained solid was dried in Vacuum Tray Drier (VTD) to obtain pure Finerenone, the compound I. Melting point: 240.8° C.; Yield: 34 gm (80.95%); HPLC chemical purity: 99.88%; Chiral purity: S-isomer 100% and R-isomer: Not detected.

### Example 21: Purification of Finerenone (Compound I)

Finerenone, the compound I (05 gm) was charged in a mixture of an industrial solvent (40 ml) and water (10 ml), then reaction mass was stirred for about 1-2 hours at a temperature of about 70-80° C. The reaction mass was cooled gradually at about 20-30° C., further stirred for about 3 hours, filtered, washed with industrial solvent. The obtained solid was dried in Air Tray Drier (ATD) at a temperature of about 50-55° C. to obtain pure finerenone, the compound I. Yield: 1.7 gm (80.38%); HPLC chemical Purity: 99.75%; Chiral Purity: S-isomer: 99.91% and R-isomer 0.09%.

1. A process for the preparation of (S)-enantiomer of 4-(4-cyano-2-methoxyphenyl)-5-ethoxy-2,8-dimethyl-1,4-dihydro-1,6-naphthyridine-3-carboxylic acid represented by a compound of formula IIIa (the compound IIIa); 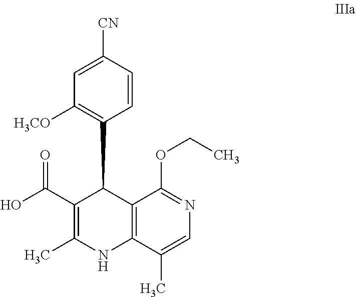 [SMILES: CCOc1ncc(C)c2c1[C@H](c1ccc(C#N)cc1OC)C(C(=O)O)=C(C)N2]
comprising the steps of:
(i) treating a racemic mixture of the compound of formula III (the compound III); 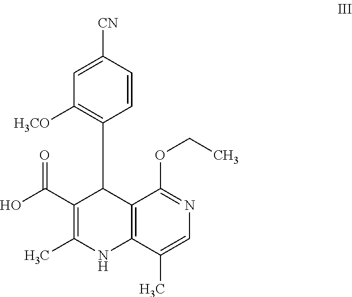 [SMILES: CCOc1ncc(C)c2c1C(c1ccc(C#N)cc1OC)C(C(=O)O)=C(C)N2]
with a chiral acid in a solvent to obtain a diastereomeric salt of the compound III with the chiral acid represented by a compound of formula IV (the compound IV); 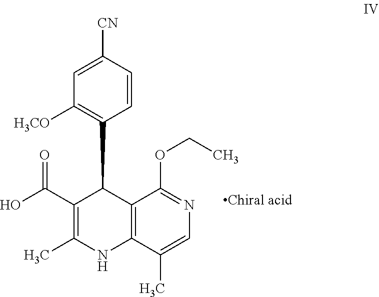 [SMILES: CCOc1ncc(C)c2c1[C@H](c1ccc(C#N)cc1OC)C(C(=O)O)=C(C)N2]
wherein the chiral acid is selected from the group consisting of L-(+) tartaric acid, D-(−) tartaric acid, (+)-dibenzoyl-D-tartaric acid, (−)-dibenzoyl-L-tartaric acid, (+)-di-p-toluoyl-D-tartaric acid, and (−)-di-p-toluoyl-L-tartaric acid; and
(ii) subjecting the compound IV obtained in the step (i) to saponification by treating the compound IV with a base or a buffer to obtain the(S)-enantiomer of 4-(4-cyano-2-methoxyphenyl)-5-ethoxy-2,8-dimethyl-1,4-dihydro-1,6-naphthyridine-3-carboxylic acid, the compound IIIa.

2. The process according to claim 1, wherein the chiral acid used in the step (i) is (+)-dibenzoyl-D-tartaric acid.

3. The process according to claim 1, wherein the solvent used in the step (i) is selected from an organic solvent, water or a mixture thereof.

4. (canceled)

5. The process according to claim 3, wherein the organic solvent is selected from the group consisting of methanol, ethanol, 1,2-ethanediol, methoxyethanol, isopropanol, n-propanol, butanol, dimethyl ether, diethyl ether, diisopropyl ether, tert-butyl methyl ether, dibutyl ether, dimethoxyethane, diethoxyethane, tetrahydrofuran (THF), dimethyltetrahydrofuran, dioxane, ethyl acetate, isopropyl acetate, isobutyl acetate, t-butyl acetate, dimethylformamide (DMF), dimethyl sulfoxide, dimethyl acetamide, and a mixture thereof.

6. The process according to claim 1, wherein in the step (ii), the compound IV is subjected to saponification by treating the compound IV with a base selected from an inorganic base or an organic base; and wherein the inorganic base is selected from sodium carbonate, potassium carbonate, sodium bicarbonate, potassium bicarbonate, sodium hydroxide, potassium hydroxide, calcium hydroxide, lithium hydroxide, sodium methoxide, or potassium methoxide; and the organic base is selected from triethylamine, diisopropylethylamine, N,N-dimethylaniline, 4-bromo-N,N-dimethylaniline, pyridine, 2-bromopyridine, 3-bromopyridine, 4-bromopyridine, 4-dimethylaminopyridine, di-tertbutyl pyridine, 2,6-di-tert-butyl-4-methylpyridine, quinoline, tri-n-butylamine, N-methylmorpholine, 2,6-lutidine, imidazole, 1,5-diazabicyclo[4.3.0]non-5-ene, 1,8-diazabicyclo[5.4.0]undec-7-ene, or 1,4-diazabicyclo[2.2.2]octane (DABCO).

7. The process according to claim 1, wherein in the step (ii), the compound IV is subjected to saponification by treating the compound IV with a buffer selected from potassium dihydrogen phosphate, dipotassium hydrogen phosphate, tripotassium phosphate, sodium dihydrogen phosphate, disodium hydrogen phosphate, tri-sodium phosphate, or hydrates thereof.

8. The process according to claim 1, wherein the step (ii) is carried out in the presence of a solvent selected from water, an organic solvent or a mixture thereof.

9. A diastereomeric salt of 4-(4-cyano-2-methoxyphenyl)-5-ethoxy-2,8-dimethyl-1,4-dihydro-1,6-naphthyridine-3-carboxylic acid with (+)-dibenzoyl-D-tartaric acid represented by a compound of formula IVa;

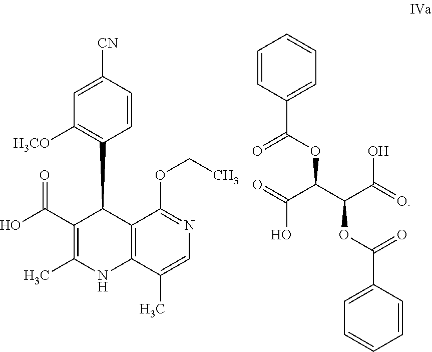

10. A process for the preparation of finerenone (the compound I); 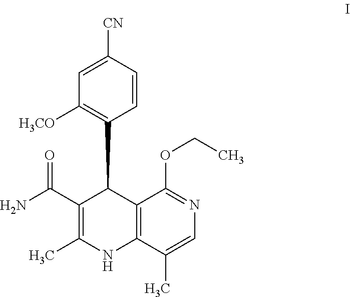 [SMILES: CCOc1ncc(C)c2c1[C@H](c1ccc(C#N)cc1OC)C(C(N)=O)=C(C)N2]
comprising the steps of;
a-1) obtaining a diastereomeric salt of 4-(4-cyano-2-methoxyphenyl)-5-ethoxy-2,8-dimethyl-1,4-dihydro-1,6-naphthyridine-3-carboxylic acid with (+)-dibenzoyl-D-tartaric acid represented by a compound of formula IVa (the compound IVa); 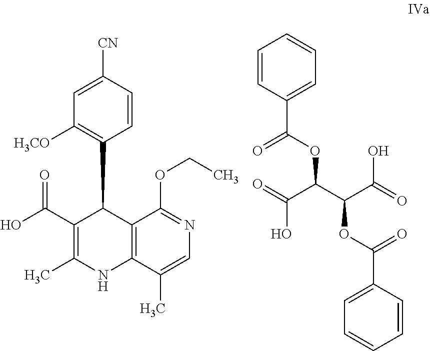
b-1) subjecting the compound IVa obtained in the step (a-1) to saponification to obtain the(S)-enantiomer of 4-(4-cyano-2-methoxyphenyl)-5-ethoxy-2,8-dimethyl-1,4-dihydro-1,6-naphthyridine-3-carboxylic acid represented by a compound of formula IIIa (the compound IIIa); 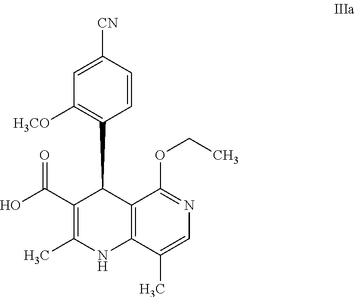 [SMILES: CCOc1ncc(C)c2c1[C@H](c1ccc(C#N)cc1OC)C(C(=O)O)=C(C)N2]
c-1) converting the compound IIIa obtained in the step (b-1) to finerenone, the compound I; and
d-1) optionally, purifying the compound I.

11. The process according to claim 10, wherein the step (a-1) for obtaining the compound IVa is carried out by treating the racemic mixture of the compound III represented by the following formula; 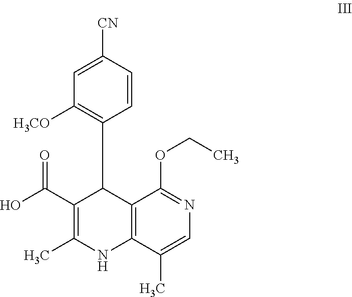 [SMILES: CCOc1ncc(C)c2c1C(c1ccc(C#N)cc1OC)C(C(=O)O)=C(C)N2]
with (+)-dibenzoyl-D-tartaric acid in the presence of a solvent selected from water, organic solvent or a mixture thereof, wherein the organic solvent is selected from methanol, ethanol, 1,2-ethanediol, methoxyethanol, isopropanol, n-propanol, ethyl acetate, isopropyl acetate, acetone, methyl ethyl ketone, cyclohexanone, toluene, xylene, acetonitrile, dimethylformamide, dimethyl sulfoxide, or a mixture thereof.

12. The process according to claim 11, wherein the saponification of the compound IVa in the step (b-1) is carried out in the presence of a base or a buffer.

13. The process according to claim 12, wherein the base is selected from an inorganic base or an organic base; and wherein the inorganic base is selected from sodium carbonate, potassium carbonate, sodium bicarbonate, potassium bicarbonate, sodium hydroxide, potassium hydroxide, calcium hydroxide, lithium hydroxide, sodium methoxide, or potassium methoxide; and the organic base is selected from triethylamine, diisopropylethylamine, N,N-dimethylaniline, 4-bromo-N,N-dimethylaniline, pyridine, 2-bromopyridine, 3-bromopyridine, 4-bromopyridine, 4-dimethylaminopyridine, di-tertbutyl pyridine, 2,6-di-tert-butyl-4-methylpyridine, quinoline, tri-n-butylamine, N-methylmorpholine, 2,6-lutidine, imidazole, 1,5-diazabicyclo[4.3.0]non-5-ene, 1,8-diazabicyclo[5.4.0]undec-7-ene, or 1,4-diazabicyclo[2.2.2]octane (DABCO).

14. The process according to claim 12, wherein the buffer is selected from potassium dihydrogen phosphate, dipotassium hydrogen phosphate, tripotassium phosphate, sodium dihydrogen phosphate, disodium hydrogen phosphate, tri-sodium phosphate, or hydrates thereof.

15. The process according to claim 10, wherein the saponification of the compound IVa in the step (b-1) is carried out in the presence of a solvent, wherein the solvent is selected from water, organic solvent or a mixture thereof, and wherein the organic solvent is selected from methanol, ethanol, 1,2-ethanediol, methoxyethanol, isopropanol, n-propanol, ethyl acetate, isopropyl acetate, acetone, methyl ethyl ketone, cyclohexanone, toluene, xylene, acetonitrile, dimethylformamide, dimethyl sulfoxide, or a mixture thereof.

16. (canceled)
